(19) United States
(12) Patent Application Publication
(10) Pub. No.: US 2018/0050087 A1
(43) Pub. Date: Feb. 22, 2018

(54) METHODS OF TREATING LIPODYSTROPHY USING FGF-1 COMPOUNDS

(71) Applicants: Salk Institute for Biological Studies, La Jolla, CA (US); Metacrine, Inc., San Diego, CA (US)

(72) Inventors: Neil McDonnell, San Diego, CA (US); Ronald M. Evans, La Jolla, CA (US); Michael Downes, San Diego, CA (US)

(73) Assignees: Salk Institute for Biological Studies, La Jolla, CA (US); Metacrine, Inc., San Diego, CA (US)

(21) Appl. No.: 15/727,164

(22) Filed: Oct. 6, 2017

Related U.S. Application Data
(63) Continuation of application No. PCT/US2016/028562, filed on Apr. 21, 2016.
(60) Provisional application No. 62/150,405, filed on Apr. 21, 2015.

(51) Int. Cl. A61K 38/18; A61K 9/00; A61K 45/06

(52) U.S. Cl. CPC A61K 38/1825; A61K 45/06; A61K 9/0019

(57) ABSTRACT

Provided herein are methods and compositions for treating lipodystrophy using fibroblast growth factor 1 (FGF-1) compounds.

### CROSS-REFERENCE TO RELATED APPLICATIONS

This application is a continuation of International Application No. PCT/US2016/028562, filed Apr. 21, 2016, which was published in English under PCT Article 21(2), which in turn which claims priority to U.S. Provisional Application No. 62/150,405, filed Apr. 21, 2015, both herein incorporated by reference.

### FIELD

The disclosure relates generally to the treatment of conditions associated with the improper production, use, or storage of fat, using FGF-1.

### BACKGROUND

Lipodystrophy is a condition characterized by problems with the way the body produces, uses, or stores fat. Lipodystrophy can be accompanied by a decrease in the hormone leptin, which, in turn, can adversely affect a patient's metabolic system, thereby leading to potentially life-threatening complications. For example, improper allocation of fat in and/or around the blood, heart, kidneys, liver, and/or pancreas can lead to insulin resistance, diabetes, high cholesterol, fatty liver disease, pancreatitis, and/or heart disease.

In some cases, lipodystrophy can develop as a small lump or dent in the skin following repeated injections of medications (i.e., insulin) at the same location. This, in turn, can potentially lead to rejection of the injected medication, a decrease in the absorption of the medication, and/or trauma.

HIV-associated lipodystrophy is a condition that occurs in between 30-50% of people who are infected with HIV and is characterized by redistribution of body fat, including lipoatrophy in subcutaneous fat of the limbs, buttocks and face; and lipohypertrophy in the abdomen, trunk, breast, and neck. Metabolic disturbances typically accompany these morphological changes and include dyslipidemia, especially hypertriglyceridemia, and disordered glucose homeostasis, typically insulin resistance. The HIV virus itself may interfere with the way that the body processes fat, thereby leading to the development of lipodystrophy.

Moreover, HIV-associated lipodystrophy may also develop as a possible side effect of antiretroviral therapy (ART) and are commonly observed in HIV patients treated with thymidine analogue nucleoside reverse transcriptase inhibitors (tNRTIs) (e.g., zidovudine (AZT) and stavudine (d4T)). ART has been shown to be associated with mitochondrial toxicity in fat cells as well as a variety of metabolic complications including, dyslipidemia, lipoatrophy, and dysregulation of glucose homeostasis. (See Hadigan, JID 198:1729-31 (2008)).

The peroxisome proliferator-activated receptor γ (PPARγ) has been a target of investigation in research regarding HIV-associated lipodystrophy because it plays a role in adipocyte cell differentiation and is preferentially expressed in subcutaneous adipose tissue. Additionally, there is also an observed clinical overlap between HIV-associated lipodistrophy and other genetic forms of lipodystrophy that have PPARγ defects. Moreover, in patients receiving tNRTIs, the levels of adipose tissue expression of PPARγ as well as PPARγ coactivator 1 (PGC1a) are low. (See Hadigan, JID 198:1729-31 (2008)).

However, to date, the results observed with the use of PPARγ agonists, such as thiazolidinedione (TZDs), have been mixed, as TZDs do not alter these expression levels of PPARγ and PGC1a. (See, e.g., Mallon et al., JID 190:1794-1803 (2008) and HIV Clin Trials 2010, 11(1):39-50). Moreover, in patients where PPARγ expression levels did increase (regardless of whether the patient received TZDs), no improvement in lipoatrophy was observed.

Accordingly, there remains a need for alternative and/or improved therapeutic approaches to lipodystrophy (e.g., HIV-associated lipodystrophy) that are more effective and have fewer adverse effects than the available approaches, including the use of TZDs.

### SUMMARY

Provided herein are compositions and methods for treating an individual suffering from or at risk for developing lipodystrophy using an FGF-1 compound. In some embodiments, pharmaceutical compositions for treating lipodystrophy comprising an FGF-1 compound are provided. For example, the FGF-1 compound may be an FGF-1 protein (such as a pre-FGF-1 protein or mature FGF-1 protein), a functional fragment of a full-length FGF-1 protein, a functional analog of FGF-1, or any combination thereof.

By way of non-limiting example, such pharmaceutical compositions may be formulated for intravenous administration, for subcutaneous administration, for intraperitoneal administration, and/or for any other method(s) of administration commonly employed in the art. The dose of the FGF-1 compound in the pharmaceutical composition may be equivalent to 0.01-50 mg FGF-1 per kg body weight of the individual, e.g., 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 5, 10, 15, 20, 25, 30, 35, 40, 45, or 50 mg/kg. If necessary, higher doses can also be utilized.

Any of the compositions described herein may also include at least one second therapeutic agent. For example, the second therapeutic agent may be a biguanide (e.g., metformin or phenformin), which may have additional effects on insulin resistance in the liver.

The lipodystrophy may be an acquired lipodystrophy (e.g., HIV-associated lipodystrophy, acquired partial lipodystrophy (Barraquer-Simons syndrome), acquired generalized lipodystrophy, centrifugal abdominal lipodystrophy (Lipodystrophia centrifugalis abdominalis infantilis), lipoatrophia annularis (Ferreira-Marquest lipoatrophia), and localized lipodystrophy) or a congenital lipodystrophy (e.g., congenital generalized lipodystrophy (Beradinelli-Seip syndrome) and familial partial lipodystrophy).

Also provided are methods of manufacturing or making a medicament for use in treating lipodystrophy comprising an FGF-1 compound as well as the use of an FGF-1 compound for treating lipodystrophy in an individual suffering therefrom or an individual at risk of developing lipodystrophy.

Also provided are methods for treating an individual having or at risk of developing lipodystrophy by administering a therapeutically effective amount of an FGF-1 compound to the individual. For example, the lipodystrophy may be an acquired lipodystrophy (e.g., HIV-associated lipodystrophy, acquired partial lipodystrophy (Barraquer-Simons syndrome), acquired generalized lipodystrophy, centrifugal abdominal lipodystrophy (Lipodystrophia centrifugalis abdominalis infantilis), lipoatrophia annularis (Ferreira-Marquest lipoatrophia), and localized lipodystrophy) or a congenital lipodystrophy (e.g., congenital generalized lipodystrophy (Beradinelli-Seip syndrome) and familial partial lipodystrophy). In one embodiment, the lipodystrophy is HIV-associated lipodystrophy.

In such methods, the FGF-1 compound may be administered orally, intranasally, intravenously, subcutaneously, intramuscularly, intradermally, and/or intraperitoneally. Any other method of administration routinely used in the art can also be utilized.

The therapeutically effective dose of the FGF-1 compound that is administered to the individual may be equivalent to 0.01-50 mg FGF-1 per kg body weight of the individual, e.g., at least 0.01 mg/kg, at least 0.1 mg/kg, such as 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 5, 10, 15, 20, 25, 30, 35, 40, 45, or 50 mg/kg. Higher therapeutically effective amounts can also be used.

In some embodiments, the method further involves administering at least one additional therapeutic agent to the individual. The at least one additional therapeutic agent can be administered at the same time (e.g., in the same composition or in a separate composition that is administered simultaneously) as the FGF-1 compound. Alternatively, the at least one additional therapeutic agent can administered at a different time than the FGF-1 compound. The at least one additional therapeutic agent may be another treatment of lipodystrophy (e.g., a biguanide) or it may be an agent that treats, improves, and/or ameliorates an associated symptom, side effect, and/or condition.

Those skilled in the art will recognize that lipodystrophy (e.g., HIV-associated lipodystrophy) is often cited as a contributing factor to nonadherence to ART regimens. Therefore, administration of an FGF-1 compound could also be used in methods of improving patient compliance or adherence to ART treatment regimens. In such methods, the FGF-1 compound and the antiretroviral agent could be administered to HIV patients simultaneously and/or in close temporal proximity in order to reduce, minimize, and/or prevent the development of HIV-associated lipodystrophy, thereby resulting in improved patient compliance with the ART treatment regimen.

Accordingly, also provided herein are methods of preventing or delaying the development of lipodystrophy (e.g., an acquired lipodystrophy such as HIV-associated lipodystrophy) in a patient receiving an antiretroviral treatment regimen, the method comprising co-administering a therapeutically effective amount of an FGF-1 compound (e.g., a functional fragment of FGF-1 or a functional analog of FGF-1) to the individual.

In such methods, the FGF-1 compound is administered intravenously, subcutaneously, or using any other administration method(s) known in the art. Likewise, in these methods, the therapeutically effective amount is between about 0.01 to about 50 mg per kg body weight and can be administered daily, twice daily, every other day, bi-weekly, weekly, or monthly. Those skilled in the art will recognize that the antiretroviral agent and the FGF-1 compound can be administered simultaneously, individually, separately, or sequentially.

Other features of the disclosure will be apparent from the following detailed description and claims.

### SEQUENCE LISTING

The nucleic and amino acid sequences are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The sequence listing generated on Oct. 6, 2017 (5.21 kb) and submitted herewith is herein incorporated by reference.

SEQ ID NOS: 1 and 2 provide an exemplary human FGF1 nucleic acid and protein sequences, respectively. Source: GenBank Accession Nos: BC032697.1 and AAH32697.1. Heparan binding residues are amino acids 127-129 and 133-134 of SEQ ID NO: 2.

SEQ ID NO: 3 provides an exemplary mature form of human FGF1 (140 aa, sometimes referred to in the art as FGF1 15-154).

### DETAILED DESCRIPTION

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising a protein” includes single or plural proteins and is considered equivalent to the phrase “comprising at least one protein.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. Dates of GenBank® Accession Nos. referred to herein are the sequences available at least as early as Apr. 21, 2016. All references and GenBank® Accession numbers cited herein are incorporated by reference.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting.

In order to facilitate review of the various embodiments of the disclosure, the following explanations of specific terms are provided:

As used herein, the term “lipodystrophy” refers to a medical condition involving abnormal or degenerative conditions of the body's adipose (fat) tissue. Lipodystrophy is also commonly referred to as “fat redistribution”. Similarly, the term “lipoatrophy” refers to the loss of fat from one area of the body. Each of these terms is used interchangeably herein to refer to conditions associated with the degeneration of or improper or abnormal distribution of adipose tissue that can be treated with FGF-1 compounds in connection with any of the methods and compositions described herein.

There are two different types of lipodystrophies: acquired lipodystrophies and congenital lipodystrophies. Examples of acquired lipodystrophies can include, for example. HIV-associated lipodystrophy, acquired partial lipodystrophy (Barraquer-Simons syndrome), acquired generalized lipodystrophy, centrifugal abdominal lipodystrophy (Lipodystrophia centrifugalis abdominalis infantilis), lipoatrophia annularis (Ferreira-Marquest lipoatrophia), and localized lipodystrophy. Examples of congenital lipodystrophies can include, for example, congenital generalized lipodystrophy (Beradinelli-Seip syndrome) and familial partial lipodystrophy.

Those skilled in the art will recognize that any of the compositions and methods described herein can be used to treat any type of lipodystrophy in a patient suffering therefrom or at risk of suffering therefrom. In one preferred embodiment, the lipodystrophy is HIV-associated lipodystrophy.

Fibroblast growth factors (FGFs) are a family of distinct polypeptide hormones that are widely expressed in developing and adult tissues. (See Baird et al., Cancer Cells, 3:239-243, 1991). FGFs play crucial roles in multiple physiological functions including angiogenesis, development, mitogenesis, pattern formation, cellular proliferation, cellular differentiation, metabolic regulation, and repair of tissue injury. (See McKeehan et al., Prog. Nucleic Acid Res. Mol. Biol. 59:135-176, 1998). The FGF family now consists of at least twenty-three members, FGF-1 to FGF-23. (See Reuss et al., Cell Tissue Res. 313:139-157 (2003).

FGFs bind to one of four FGF receptors (FGFRs), which are known as FGFR1-4. The receptor binding specificity of each FGF is distinct and can also depend on the particular isoform of the FGFR. For example FGFR1 has at least 3 isoforms that result in different splice variants in the third Ig-like domain. (See Lui et al. (2007) Cancer Res. 67:2712). FGF signaling is also determined by the tissue specificity of the receptor and the receptor isoform. FGF-1 can bind to all FGFRs, but is reported to be internalized only upon binding to FGFR1 and FGFR4. A review of FGF-FGFR specificities can be found, e.g., in Sorensen et al. (2006) J Cell Science 119:4332, which is herein incorporated by reference.

FGF-1 (also referred to as acidic FGF) (OMIM 13220) is a secreted protein that binds heparin (e.g., heparin sulfate) and to FGF receptor family members 1 and 4. The human precursor protein is 155 amino acids in length (SEQ ID NO: 2). FGF-1 protein and nucleic acid sequences are publically available, for example at SwissProt accession number P05230.1 or from GenBank® sequence database (e.g., Accession Nos. NP_00791 and NP_034327 provide exemplary FGF1 protein sequences, while Accession Nos. NM_000800 and NM_010197 provide exemplary FGF1 nucleic acid sequences). Likewise, the polypeptide and coding sequences of FGF-1 are known for a number of animals and are publically available from the NCBI website. The mature form of human FGF1 does not include the 14 N-terminal amino acids of the precursor protein (SEQ ID NO: 3, it is 140 aa, sometimes referred to in the art as FGF-1 15-154). Thus, as used herein, “FGF-1” refers to both the precursor and mature forms of FGF-1.

Similarly, the term “FGF-1 compound” refers to FGF-1 (e.g., SEQ ID NO: 2 or 3 or a species homolog thereof) or to a variant thereof that retains at least one FGF-1 activity (e.g., retains at least 10%, 20%, 30%, 40%, 50%, 60%, 70%, 80%, 90%, 95% or higher percent activity compared to FGF-1). The FGF-1 compound may be generated, isolated, and/or purified by any means known in the art. For standard recombinant methods, see Sambrook et al., Molecular Cloning: A Laboratory Manual, Cold Spring Harbor Laboratory Press, NY (1989); Deutscher, Methods in Enzymology 182: 83-9 (1990); Scopes, Protein Purification: Principles and Practice, Springer-Verlag, NY (1982).

As used herein, the terms “FGF-1” and “FGF-1 compound” refer to naturally-occurring, isolated, recombinant, or synthetically-produced proteins as well as allelic variants and species homologs.

Non-limiting examples of FGF-1 variants include fragments (i.e., functional fragments), portions, modified forms, analogs (i.e., functional analogs), expression vectors for stable or transient expression of FGF-1 in a cell, and/or proteins or polypeptides having substantial identity (e.g., at least 80%, 85%, 90%, 991%, 92%, 93%, 94%, 5%, 96%, 97%, 98%, 99%, or 100% amino acid identity) to FGF-1 (e.g., such sequence identity to SEQ ID NO: 2 or 3), where the non-identities represent conservative substitutions or additions or deletions that do not substantially change the at least one activity of FGF-1.

As used herein, the terms “identical” or percent “identity,” in the context of two or more nucleic acids or two or more polypeptides, refer to two or more sequences or subsequences that are the same or have a specified percentage of nucleotides, or amino acids, that are the same (e.g., about 60% identity, such as 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. (See, e.g., the NCBI web site at ncbi.nlm.nih.gov/BLAST). Such sequences are then said to be “substantially identical.” This definition also refers to, or may be applied to, the compliment of a nucleotide test sequence. The definition also includes sequences that have deletions and/or additions, as well as those that have substitutions. Algorithms can account for gaps and the like. Identity generally exists over a region that is at least about 25 amino acids or nucleotides in length, or over a region that is 50-100 amino acids or nucleotides in length.

“FGF-1 activities” include, for example, binding heparin, FGFR1, and FGFR4, and increasing expression of GLUT1 and/or GLUT4. Other FGF-1 activities also known in the art. (See, e.g., WO2011/130729, which is herein incorporated by reference in its entirety).\

A “functional FGF-1 fragment” is a protein having less than the full length sequence of FGF-1 (e.g., less than 155 amino acids of the precursor protein (e.g., a fragment of SEQ ID NO: 2) or less than 140 aa of the mature protein (e.g., a fragment of SEQ ID NO: 3)) but that retains at least 25%, at least 50%, at least 80%, at least 90%, or at least 95% activity of at least one FGF-1 activity. Examples of functional fragments may include amino acids 14-135, 14-155, 13-135, 14-154, etc. of the precursor protein (e.g., of SEQ ID NO: 2) or amino acids 5-140, 6-140, 7-140, 8-140, 9-140, 10-140, 11-140, 12-140, 13-140, 14-140, or 15-140 of the mature protein (e.g., of SEQ ID NO: 3). The functional FGF-1 fragment can have an amino acid sequence of any length up to the full length FGF polypeptide sequence, e.g., 30, 50, 10-140, 11-140, 12-140, 13-140, 14-140, 15-140, 20-140, 30-140, 50-140, 50-80, 50-100, 120-150, 100-150, or more than 100 contiguous amino acids of an FGF-1 protein sequence (e.g., of SEQ ID NO: 2 or 3). Likewise, the functional FGF fragment may be at least 80%, at least 85%, at least 90%, at least 91%, at least 92%, at least 93%, at least 94%, at least 95%, at least 96%, at least 97%, at least 98%, or at least 100% identical to FGF-1 over the covered portion of the full length sequence (e.g., over 50-150 amino acids). For example, the functional FGF-1 fragment may have greater than 90%, e.g., at least 95%, at least 96%, at least 97%, at least 98%, at least 99% or higher % identity to amino acids 1-140 of mature FGF-1 (e.g., of SEQ ID NO: 3). In some embodiments, the non-identities represent conservative substitutions or additions or deletions that do not substantially change the activity of the FGF-1 fragment.

In additional embodiments, a functional FGF-1 fragment has about 1, 2, 3, 4, 5, 6, 7, 8, 9, 10, 11, 12, 13, 14, 15, 20, or more mutations. A functional FGF-1 fragment can have about 1 or more mutation. A functional FGF-1 fragment can have about 2 or more mutations. A functional FGF-1 fragment can have about 3 or more mutations. A functional FGF-1 fragment can have about 4 or more mutations. A functional FGF-1 fragment can have about 5 or more mutations. A functional FGF-1 fragment can have about 6 or more mutations. A functional FGF-1 fragment can have about 7 or more mutations. A functional FGF-1 fragment can have about 8 or more mutations. A functional FGF-1 fragment can have about 9 or more mutations. A functional FGF-1 fragment can have about 10 or more mutations. A functional FGF-1 fragment can have about 11 or more mutations. A functional FGF-1 fragment can have about 12 or more mutations. A functional FGF-1 fragment can have about 13 or more mutations. A functional FGF-1 fragment can have about 14 or more mutations. A functional FGF-1 fragment can have about 15 or more mutations. A functional FGF-1 fragment can have about 16 or more mutations. A functional FGF-1 fragment can have about 17 or more mutations. A functional FGF-1 fragment can have about 18 or more mutations. A functional FGF-1 fragment can have about 19 or more mutations. A functional FGF-1 fragment can have about 20 or more mutations. The mutation can be a missense mutation or a nonsense mutation. The mutation can be an insertion or a deletion. The insertion can be a duplication or an insertion of a repeating sequence. The mutation can also be a conserved substitution, additional or deletion.

FGF-1 compounds can also be modified, e.g., to improve stability (e.g., PEGylated or including non-naturally occurring amino acids) or its pharmacological profile. Chemical modifications include, e.g., adding chemical moieties, creating new bonds, and removing chemical moieties. Modifications at amino acid side groups include acylation of lysine ε-amino groups, N-alkylation of arginine, histidine, or lysine, alkylation of glutamic or aspartic carboxylic acid groups, and deamidation of glutamine or asparagine. Modifications of the terminal amino group include the des-amino, N-lower alkyl, N-di-lower alkyl, and N-acyl modifications. Modifications of the terminal carboxy group include the amide, lower alkyl amide, dialkyl amide, and lower alkyl ester modifications.

Compounds that can improve the pharmacological profile of the FGF-1 compound include, for example, water soluble polymers, such as PEG, PEG derivatives, polyalkylene glycol (PAG), polysialyic acid, hydroxyethyl starch, peptides (e.g., Tat (from HIV), Ant (from the Drosophila antennapedia homeotic protein), or poly-Arg), and small molecules (e.g., lipophilic compounds such as cholesterol or DAG).

In some embodiments, the FGF-1 compound can be linked to a heparin molecule, which can improve the stability of FGF-1, and prevent interaction with heparin in vivo. Linking heparin to FGF-1 ensures that more of the modified FGF-1 remains in circulation than it would without the heparin modification.

The term “functional FGF-1 analog” refers to a modified or synthetic (e.g., peptidomimetic) form of FGF-1 that retains at least 25%, at least 50%, at least 80%, at least 85%, at least 90%, at least 95%, at least 96%, at least 97%, at least 98%, or at least 99% activity of at least one FGF-1 activity. Examples of FGF-1 analogs that retain heparin-binding activity are disclosed in WO2006/093814, which is incorporated herein by reference. The FGF-1 analog can include non-naturally occurring amino acids, or modified amino acids, such as those that improve the stability (in storage or in vivo) or the pharmacological properties (tissue profile, half-life, etc.) of the protein. The functional FGF-1 analog can also be a functional FGF-1 variant, as defined herein.

The term “nucleic acid” refers to deoxyribonucleotides or ribonucleotides and polymers thereof in either single- or double-stranded form, and complements thereof. The term “polynucleotide” refers to a linear sequence of nucleotides. The term “nucleotide” typically refers to a single unit of a polynucleotide (i.e., a monomer). Nucleotides can be ribonucleotides, deoxyribonucleotides, or modified versions thereof. Examples of polynucleotides contemplated herein include single and double stranded DNA, single and double stranded RNA (including siRNA), and hybrid molecules having mixtures of single and double stranded DNA and RNA.

As used herein, the words “complementary” or “complementarity” refer to the ability of a nucleic acid in a polynucleotide to form a base pair with another nucleic acid in a second polynucleotide. For example, the sequence A-G-T is complementary to the sequence T-C-A. Complementarity may be partial, in which only some of the nucleic acids match according to base pairing, or complete, where all the nucleic acids match according to base pairing.

The words “protein”, “peptide”, and “polypeptide” are used interchangeably herein to denote an amino acid polymer or a set of two or more interacting or bound amino acid polymers. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer.

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, e.g., an a carbon that is bound to a hydrogen, a carboxyl group, an amino group, and an R group, e.g., homoserine, norleucine, methionine sulfoxide, methionine methyl sulfonium. Such analogs may have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions similarly to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As used herein, the term “recombinant” indicates that the cell, nucleic acid, protein or vector has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “heterologous” indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

Those skilled in the art will recognize that any of the FGF-1 compounds described herein can be expressed recombinantly using routine techniques in the field of recombinant genetics. Standard techniques are used for cloning, DNA and RNA isolation, amplification and purification. Generally enzymatic reactions involving DNA ligase, DNA polymerase, restriction endonucleases and the like are performed according to the manufacturer's specifications. Basic texts disclosing the general methods of use include Sambrook and Russell eds. (2001) Molecular Cloning: A Laboratory Manual, 3rd edition; the series Ausubel et al. eds. (2007 with updated through 2010) Current Protocols in Molecular Biology, among others known in the art.

To obtain high level expression of a nucleic acid sequence, such as a nucleic acid sequence encoding an FGF-1 compound (e.g., SEQ ID NO: 1), a nucleic acid sequence that encodes a polypeptide sequence can be subcloned into an expression vector that is subsequently transfected into a suitable host cell. The expression vector typically contains a strong promoter or a promoter/enhancer to direct transcription, a transcription/translation terminator, and for a nucleic acid encoding a protein, a ribosome binding site for translational initiation. The promoter is operably linked to the nucleic acid sequence encoding a polypeptide or a subsequence thereof.

Any of the conventional vectors used for expression in eukaryotic or prokaryotic cells may be used to transport the genetic information into the cell. Standard bacterial expression vectors include plasmids such as pBR322 based plasmids, pSKF, pET23D, and fusion expression systems such as GST and LacZ.

Epitope tags can also be added to the recombinant polypeptides to provide convenient methods of isolation (e.g., His tags). In some case, enzymatic cleavage sequences (e.g., Met-(His)_{g}-Ile-Glu-Gly-Arg which form the Factor Xa cleavage site) are added to the recombinant polypeptides. Bacterial expression systems for expressing the polypeptides are available in, e.g., E. coli, Bacillus sp., and Salmonella (Palva et al., Gene 22:229-235 (1983); Mosbach et al., Nature 302:543-545 (1983). Kits for such expression systems are commercially available. Eukaryotic expression systems for mammalian cells, yeast, and insect cells are well known in the art and are also commercially available.

Standard transfection methods can be used to produce cell lines that express large quantities of polypeptides, which are then purified using standard techniques (see, e.g., Colley et al., J. Biol. Chem., 264:17619-17622 (1989); Guide to Protein Purification, in Methods in Enzymology, vol. 182 (Deutscher, ed., 1990)). Transformation of cells is performed according to standard techniques (see, e.g., Morrison, J. Bact., 132:349-351 (1977); Clark-Curtiss & Curtiss, Methods in Enzymology, 101:347-362 (Wu et al., eds, 1983). For example, any of the well-known procedures for introducing foreign nucleotide sequences into host cells may be used. These include the use of calcium phosphate transfection, polybrene, protoplast fusion, electroporation, liposomes, microinjection, plasma vectors, and viral vectors (see, e.g., Sambrook and Russell eds. (2001) Molecular Cloning: A Laboratory Manual, 3rd edition).

FGF-1 can be purified to substantial purity by standard techniques known in the art, including, for example, extraction and purification from inclusion bodies, size differential filtration, solubility fractionation (i.e., selective precipitation with such substances as ammonium sulfate); column chromatography, immunopurification methods, etc.

Any of the FGF-1 compounds described herein can also be chemically synthesized using any known methods including, e.g., solid phase synthesis (see, e.g., Merrifield, J. Am. Chem. Soc., 85:2149-2154 (1963) and Abelson et al., Methods in Enzymology, Volume 289: Solid-Phase Peptide Synthesis (1st ed. 1997)). Polypeptide synthesis can be performed using manual techniques or by automation. Automated synthesis can be achieved, for example, using Applied Biosystems 431A Peptide Synthesizer (Perkin Elmer). Alternatively, various fragments of the polypeptide (and any modified amino acids) can be chemically synthesized separately and then combined using chemical methods to produce the full length polypeptide. The sequence and mass of the polypeptides can be verified by GC mass spectroscopy. Once synthesized, the polypeptides can be modified, for example, by N-terminal acetyl- and C-terminal amide-groups as described above. Synthesized FGF-1 polypeptides can be further isolated by HPLC to a purity of at least about 80%, such as at least 90%, or at least 95%.

FGF-1 variants can include “conservatively modified variants” of both amino acid and nucleic acid sequences. With respect to particular nucleic acid sequences, conservatively modified variants include those nucleic acids which encode identical or essentially identical amino acid sequences, or, where the nucleic acid does not encode an amino acid sequence, to essentially identical or associated, e.g., naturally contiguous, sequences. Because of the degeneracy of the genetic code, a large number of functionally identical nucleic acids encode most proteins. The terms “silent variations” or “silent substitutions” and the like refer to changes to codons that do not alter the encoded polypeptide.

Other non-limiting examples of conservatively modified variants include individual substitutions, deletions or additions to a nucleic acid, peptide, polypeptide, or protein sequence which alters, adds or deletes a single amino acid or a small percentage of amino acids in the encoded sequence, where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles. The following amino acids are typically conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M).

“Biopsy” or “biological sample from a patient” as used herein refer to samples obtained from a patient having, or suspected of having, lipodystrophy. In some embodiments, the biopsy is a blood sample, which can be separated into blood components (plasma, serum, white blood cells, red blood cells, platelets, etc.). In some embodiments, the sample is a tissue biopsy, such as needle biopsy, fine needle biopsy, surgical biopsy, etc. Tissue samples can be obtained from adipose, muscle, liver, etc.

A “biological sample” or “cellular sample” can be obtained from a patient (e.g., a biopsy), from an animal (e.g., an animal model), or from cultured cells (e.g., a cell line or cells removed from a patient and grown in culture for observation). Biological samples include tissues (e.g., adipose tissue) and bodily fluids (e.g., blood, blood fractions, lymph, saliva, urine, feces, etc.).

“Subject,” “patient,” “individual” and similar terms are used interchangeably herein to refer to mammals such as humans and non-human primates, pigs, sheep, cows, dogs, cats, rodents and the like. The term does not necessarily indicate that the subject has been diagnosed with lipodystrophy, but typically refers to an individual under medical supervision or a patient at risk of developing lipodystrophy (e.g., an HIV patient). A patient can be an individual that is seeking treatment, monitoring, adjustment or modification of an existing therapeutic regimen, etc. The terms can also refer to an individual that has been diagnosed, is currently following a therapeutic regimen, and/or is at risk of developing lipodystrophy.

A “control” condition or sample refers to a sample that serves as a reference, usually a known reference, for comparison to a test condition or sample. For example, a test sample can represent a patient sample, while a control can represent a sample from an individual known to have lipodystrophy, or from an individual that is known to not have lipodystrophy. In another example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of benefit and/or side effects). One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

Any of the FGF-1 compounds described (e.g., SEQ ID NO: 2 or 3) herein can be used and formulated into any of a number of pharmaceutical compositions, including those described in the United States Pharmacopeia (U.S.P.), Goodman and Gilman's The Pharmacological Basis of Therapeutics, 10^{th} Ed., McGraw Hill, 2001; Katzung, Ed., Basic and Clinical Pharmacology, McGraw-Hill/Appleton & Lange, 8^{th} ed., Sep. 21, 2000; Physician's Desk Reference (Thomson Publishing; and/or The Merck Manual of Diagnosis and Therapy, 18^{th} ed., 2006, Beers and Berkow, Eds., Merck Publishing Group; or, in the case of animals, The Merck Veterinary Manual, 9^{th} ed., Kahn Ed., Merck Publishing Group, 2005).

Likewise, the compositions disclosed herein can be administered by any means known in the art. For example, compositions may include administration to a subject intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intrathecally, subcutaneously, subconjunctival, intravesicularlly, mucosally, intrapericardially, intraumbilically, intraocularly, orally, locally, by inhalation, by injection, by infusion, by continuous infusion, by localized perfusion, via a catheter, via a lavage, in a creme, or in a lipid composition. Administration can be local, e.g., to adipose tissue or to the liver, or systemic.

Solutions of the active compounds as free base or pharmacologically acceptable salt can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations can contain a preservative to prevent the growth of microorganisms.

For parenteral administration in an aqueous solution, for example, the solution should be suitably buffered and the liquid diluent first rendered isotonic with sufficient saline or glucose. Aqueous solutions, in particular, sterile aqueous media, are especially suitable for intravenous, intramuscular, subcutaneous and intraperitoneal administration. For example, one dosage can be dissolved in 1 ml of isotonic NaCl solution and either added to 1000 ml of hypodermoclysis fluid or injected at the proposed site of infusion.

Sterile injectable solutions can be prepared by incorporating the active compounds or constructs in the required amount in the appropriate solvent followed by filtered sterilization. Generally, dispersions are prepared by incorporating the various sterilized active ingredients into a sterile vehicle which contains the basic dispersion medium. Vacuum-drying and freeze-drying techniques, which yield a powder of the active ingredient plus any additional desired ingredients, can be used to prepare sterile powders for reconstitution of sterile injectable solutions. The preparation of more, or highly, concentrated solutions for direct injection is also contemplated. DMSO can be used as solvent for extremely rapid penetration, delivering high concentrations of the active agents to a small area.

Heparin can interfere with FGF-1 circulation when the FGF-1 compound is not administered intravenously. For non-i.v. administration (e.g., subcutaneous administration) the FGF-1 compound can be linked to a heparin molecule, or another compound that interferes with FGF-1 binding to heparin. The FGF-1-heparin interaction in vivo reduces the amount of circulating FGF-1, and the duration of the therapeutic effect. Thus, in some embodiments, pharmaceutical compositions comprising an FGF-1 compound (e.g., SEQ ID NO: 2 or 3) linked to heparin are provided.

Pharmaceutical compositions can be delivered via intranasal or inhalable solutions or sprays, aerosols or inhalants. Nasal solutions can be aqueous solutions designed to be administered to the nasal passages in drops or sprays. Nasal solutions can be prepared so that they are similar in many respects to nasal secretions. Thus, the aqueous nasal solutions usually are isotonic and slightly buffered to maintain a pH of 5.5 to 6.5. In addition, antimicrobial preservatives, similar to those used in ophthalmic preparations, and appropriate drug stabilizers, if required, may be included in the formulation.

Oral formulations can include excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders. In some embodiments, oral pharmaceutical compositions will comprise an inert diluent or assimilable edible carrier, or they may be enclosed in hard or soft shell gelatin capsule, or they may be compressed into tablets, or they may be incorporated directly with the food of the diet. For oral therapeutic administration, the active compounds may be incorporated with excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 75% of the weight of the unit, such as between 25-60%. The amount of active compounds in such compositions is such that a suitable dosage can be obtained.

In some embodiments, the FGF-1 compound is administered using a gene therapy construct, e.g., as described in Nikol et al. (2008) Mol. Ther. Thus, an individual can be treated for lipodystrophy by administering to the individual an expression vector comprising a sequence that codes for a FGF-1 compound (e.g., SEQ ID NO: 1). Similarly, the methods of inducing fatty liver in an animal can rely on administration of an expression vector, in this case, an expression vector encoding an antisense construct specific for FGF-1.

In some cases, a polynucleotide encoding FGF-1 (e.g., SEQ ID NO: 1) is introduced into a cell in vitro and the cell is subsequently introduced into a subject. In some cases, the cells are first isolated from the subject and then re-introduced into the subject after the polynucleotide is introduced. In some embodiments, FGF-1-encoding polynucleotides or FGF-1 inhibitory polynucleotides are introduced directly into cells in the subject in vivo.

Conventional viral and non-viral based gene transfer methods can be used to introduce nucleic acids encoding FGF-1 polypeptides in mammalian cells or target tissues. Such methods can be used to administer nucleic acids encoding FGF-1 polypeptides, or FGF-1 inhibitory polynucleotides to cells in vitro. In some embodiments, such polynucleotides are administered for in vivo or ex vivo gene therapy uses. Non-viral vector delivery systems include DNA plasmids, naked nucleic acid, and nucleic acid complexed with a delivery vehicle such as a liposome. Viral vector delivery systems include DNA and RNA viruses, which have either episomal or integrated genomes after delivery to the cell. For a review of gene therapy procedures, see Anderson, Science 256:808-813 (1992); Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Böhm (eds) (1995); and Yu et al., Gene Therapy 1:13-26 (1994).

Methods of non-viral delivery of nucleic acids encoding engineered polypeptides include lipofection, microinjection, biolistics, virosomes, liposomes, immunoliposomes, polycation or lipid:nucleic acid conjugates, naked DNA, artificial virions, and agent-enhanced uptake of DNA. Lipofection is described, e.g., in U.S. Pat. No. 5,049,386, U.S. Pat. No. 4,946,787; and U.S. Pat. No. 4,897,355, and lipofection reagents are sold commercially (e.g., Transfectam™ and Lipofectin™). Cationic and neutral lipids that are suitable for efficient receptor-recognition lipofection of polynucleotides include those of Felgner, WO 91/17424, WO 91/16024. Delivery can be to cells (ex vivo administration) or target tissues (in vivo administration). The preparation of lipid:nucleic acid complexes, including targeted liposomes such as immunolipid complexes, is known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

RNA or DNA viral based systems can be used to target the delivery of polynucleotides carried by the virus to specific cells in the body and deliver the polynucleotides to the nucleus. Viral vectors can be administered directly to patients (in vivo) or they can be used to transfect cells in vitro. In some cases, the transfected cells are administered to patients (ex vivo). Conventional viral based systems for the delivery of polypeptides could include retroviral, lentivirus, adenoviral, adeno-associated and herpes simplex virus vectors for gene transfer. Viral vectors are currently the most efficient and versatile method of gene transfer in target cells and tissues. Integration in the host genome is possible with the retrovirus, lentivirus, and adeno-associated virus gene transfer methods, often resulting in long term expression of the inserted transgene, and high transduction efficiencies.

Without being bound by theory, it is proposed that because FGF-1 acts downstream of PPARγ/PGC1a, it can overcome the limitations observed with TZDs in patients with lipodystrophy (e.g., HIV-associated lipodystrophy). Additionally, by reducing or preventing the development or occurrence of HIV-associated lipodystrophy, co-administration of an FGF-1 compound could also be used in methods of improving patient compliance or adherence to ART treatment regimens.

Thus, provided herein are methods of treating, preventing, and/or ameliorating lipodystrophy in a subject in need thereof. The course of treatment can be determined on an individual basis depending on the particular characteristics of the subject. The treatment can be administered to the subject on a daily, twice daily, every other day, every third day, bi-weekly, weekly, monthly or any applicable basis that is therapeutically effective. The treatment can be administered alone or in combination with at least one additional therapeutic agent, e.g., targeting the same disorder or a related symptom. The additional agent can be administered simultaneously with the FGF-1 compound, at a different time, or on an entirely different therapeutic schedule (e.g., the FGF-1 compound can be administered daily, while the additional agent is weekly).

Because lipodystrophy (e.g., HIV-associated lipodystrophy) is often cited as a contributing factor to nonadherence to ART regimens, co-administration of an FGF-1 compound could also be used to improve patient compliance and/or adherence to ART treatment regimens. Those skilled in the art will recognize that the FGF-1 compound and the antiretroviral agent could be administered to HIV patients simultaneously, sequentially, separately, and/or in close temporal proximity to the antiretroviral agent in order to reduce, minimize, and/or prevent the development of HIV-associated lipodystrophy. Such methods would result in improved patient compliance with the ART treatment regimen. Determination of the exact dosing schedule or the FGF-1 compound and the antiretroviral agent is within the routine level of skill in the art.

The suitability of a particular route of administration will depend in part on the pharmaceutical composition, its components, and the disorder being treated. Parenteral administration is often effective for systemic treatment.

The terms “therapy,” “treatment,” “amelioration”, “improvement” and the like refer to any reduction or change in the severity of symptoms. In the case of treating lipodystrophy, the term can refer to increasing, restoring, and/or redistributing adipose tissue in the individual. As used herein, the terms “treat” and “prevent” are not intended to be absolute terms. For example, treatment can refer to any delay in onset, amelioration of symptoms, improvement in patient survival, increase in survival time or rate, etc. The effect of treatment can be compared to an individual or pool of individuals not receiving the treatment, or to the same patient prior to treatment or at a different time during treatment. In some aspects, the severity of disease is reduced by at least 10%, as compared, e.g., to the individual before administration or to a control individual not undergoing treatment. In some aspects the severity of disease is reduced by at least 25%, at least 50%, at least 75%, at least 80%, or at least 90%, or in some cases, no longer detectable using standard diagnostic techniques.

The term “diagnosis” refers to a relative probability a subject has lipodystrophy. Similarly, the term “prognosis” refers to a relative probability that a certain future outcome may occur in the subject (i.e., the likelihood that an individual will develop lipodystrophy). Prognosis can also refer to the likely severity of the disease (e.g., severity of symptoms, rate of functional decline, survival, etc.). These terms are not intended to be absolute, as will be appreciated by any one of skill in the field of medical diagnostics.

The terms “effective amount,” “effective dose,” “dosage”, “dose”, “therapeutically effective amount,” “therapeutic dosage”, and the like refer to that amount of the therapeutic agent sufficient to ameliorate a disorder, as described above. For example, for the given parameter, a therapeutically effective amount will show an increase or decrease of therapeutic effect at least 5%, at least 10%, at least 15%, at least 20%, at least 25%, at least 40%, at least 50%, at least 60%, at least 75%, at least 80%, at least 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control. The effective amount of an FGF-1 compound can vary depending on co-administration of other therapeutics or metabolic profile of the individual (among other factors such as age, severity of disease, etc.).

The dosage of a therapeutic agent administered to a patient will vary depending on a wide range of factors. For example, it may be necessary to provide substantially larger doses to humans than to smaller animals. The dosage can depend upon the size, age, sex, weight, medical history and condition of the patient, use of other therapies, the potency of the substance being administered, and the frequency of administration.

In some embodiments, the dose of the FGF-1 compound can be equivalent to 0.005-50 mg FGF-1 protein per kg body weight (e.g., at least 0.005, at least 0.01, at least 0.05, at least 0.1, at least 0.5, such as 0.005, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 2, 5, 10, 15, 20, 25, 30, 35, 40, 45, or 50 mg FGF-1 protein per kg body weight). In some cases, higher therapeutically effective amounts of the FGF-1 compounds can be employed. One of skill will understand and be able to adjust to situations where the FGF-1 compound is smaller (e.g., a functional FGF-1 fragment) or larger (e.g., a modified FGF-1 polypeptide) than FGF-1.

Determination of the appropriate dosages and the timing of administration is within the routine level of skill in the art. Thus, those skilled in the art can determine appropriate dosing by administering relatively small amounts and monitoring the patient for therapeutic effect. If necessary, incremental increases in the dose can be made until the desired results are obtained. Generally, treatment is initiated with smaller dosages which may be less than the optimum dose of the therapeutic agent. Thereafter, the dosage is increased by small increments until the optimum effect under circumstances is reached. The total daily dosage can be divided and administered in portions during the day if desired.

The pharmaceutical preparation can be packaged or prepared in unit dosage form. In such form, the preparation is subdivided into unit doses containing appropriate quantities of the active component, e.g., according to the dose of the therapeutic agent. The unit dosage form can be a packaged preparation, the package containing discrete quantities of preparation. The composition can, if desired, also contain other compatible therapeutic agents.

In some embodiments, the FGF-1 compound is co-administered with at least one additional therapeutic agent, e.g., another therapeutic agent for treating lipodystrophy, or a therapeutic agent to address associated symptoms. Therapeutic agents commonly used for lipodystrophy include, but are not limited to, Egrifta® (tesamorelin), hormones (e.g., testosterone and/or human growth hormone), Glucophage® (metformin), statins, and/or metreleptin.

The combination of an FGF-1 compound with another therapeutic agent can result in a synergistic effect with enhanced efficacy in the treatment of lipodystrophy and related conditions. The synergy allows for reduced dosages of the active agents in combination as compared to the dosages for either active individually. The reduced dosage can help reduce any side effects that may appear.

Accordingly, in combination therapy, the effective amount of the additional (second) therapeutic agent and the effective amount of the FGF-1 compound are together effective to reduce the symptoms/effects of lipodystrophy.

### EXAMPLES

These examples are provided for illustrative purposes only and not to limit the scope of the claims provided herein.

### Example 1—FGF Expression and Purification

FGF1 variants and FGF1 wild-type are expressed from pET28(+)/BL21(DE3) E. coli expression system. Protein expression from a 0.5 L culture is induced by 0.5 mM isopropyl-β-D-thio-galactoside (IPTG) at OD600 between 0.6-1.0, with simultaneous reduction of incubation temperature from 37° C. to 20° C., followed by overnight incubation. Expression cultures are harvested and lysed in 20 mM Tris buffer pH 7.4 with 0.5 M NaCl, 0.1 mg/mL phenylmethylsulfonyl fluoride (PMSF), 1 mM ethylenediaminetetraacetic acid (EDTA), 0.25 mg/mL lysozyme (Sigma) and supplemented with Benzonase® Nuclease (Millipore). The expressed protein mutants are purified by sequential chromatography on heparin sepharose resin (GE Healthcare Life Sciences) and SP sepharose resin (GE Healthcare Life Sciences).

### Example 2—Glucose Level Study

Sixty eight week old ob/ob male mice (B6.Cg-Lep^{ob}/J, Jackson lab) are randomized into three groups and treated with daily subcutaneous (s.c.) injection of a FGF variant, a control drug, or vehicle. Blood glucose levels are measured in fed animals one hour after treatment.

Glucose tolerance test are conducted after overnight fasting. Mice are intraperitoneally (i.p.) injected with about 1 g of glucose/kg body weight, and blood glucoses are monitored at 0, 10, 20, 30, 60, or 120 min post injection. Insulin tolerance tests are conducted after overnight fasting. Mice are injected i.p. with 0.5 U of insulin/kg body weight (Humulin R; Eli Lilly) and blood glucose are monitored at 0, 10, 20, 30, 60, or 120 min post injection.

### Example 3—Clinical Trial Study on the Safety and Efficacy of a FGF Variant for Lipodystrophy

### Study Type: Interventional

### Study Design:

Allocation: Randomized

Endpoint Classification: Safety/Efficacy Study

Intervention Model: Parallel Assignment

Masking: Open Label

Primary Purpose: Treatment

### Primary Outcome Measures:

Percent of liver fat pre/post challenge with daily FGF1 variant [Time Frame: 16 weeks] [Designated as safety issue: No]

### Secondary Outcome Measures:

Fine needle aspiration of fat pre/post with daily FGF1 variant [Time Frame: 16 weeks] [Designated as safety issue: No]

### Eligibility:

### Ages Eligible for Study: 18 Years to 80 Years

### Genders Eligible for Study: Both

### Accepts Healthy Volunteers: No

### Criteria

### Inclusion Criteria:

Participants must be 18 years of age or older of all racial and ethnic origins, and capable of giving informed consent;

On stable HAART for at least the last 3 months prior to entering the study;

Practitioner diagnosed lipodystrophy as defined by: aHAL decreased subcutaneous fat in the limbs with prominent veins, loss of buttock fat or facial atrophy

hHAL: fat accumulation in abdomen and/or dorsocervical region.

### Exclusion Criteria:

Women of child bearing potential

Prior history of CHF;

Prior history of macular retinal edema;

Prior history of spontaneous bone fracture;

Diabetics receiving oral/injected/inhaled diabetic agents or individuals with a fasting blood glucose value greater than or equal to 140 within the last 90 days;

Current active opportunistic infections for example:
a. PCP pneumonia
b. Neuropathy
c. Thrush
d. Systemic KS (Kaposi sarcoma)
e. MAC (Mycobacterium Avium complex)
f. Histoplasmosis
g. Coccidioidomycosis

Planning to discontinue HAART;

Current diagnosis of cancer or receiving chemotherapy;

Systemic steroid use during the prior 6 months;

Hepatitis C+ or previous diagnosis of cirrhosis; or

Liver Function Studies great than or equal to triple of normal values.

The foregoing description has been presented only for the purposes of illustration and is not intended to limit the invention to the precise form disclosed, but by the claims appended hereto.

What is claimed is:

1. A method for treating an individual suffering from or at risk from developing lipodystrophy comprising administering a therapeutically effective amount of an FGF-1 compound to the individual.

2. A method of preventing or delaying the development of lipodystrophy in an individual receiving an antiretroviral treatment regimen, comprising co-administering a therapeutically effective amount of an FGF-1 compound and the antiretroviral treatment regimen to the individual.

3. The method of claim 1, wherein the lipodystrophy is an acquired lipodystrophy.

4. The method of claim 3, wherein the acquired lipodystrophy HIV-associated lipodystrophy, acquired partial lipodystrophy (Barraquer-Simons syndrome), acquired generalized lipodystrophy, centrifugal abdominal lipodystrophy (Lipodystrophia centrifugalis abdominalis infantilis), lipoatrophia annularis (Ferreira-Marquest lipoatrophia), or localized lipodystrophy.

5. The method of claim 3, wherein the acquired lipodystrophy is HIV-associated lipodystrophy.

6. The method of claim 1, wherein the lipodystrophy is a congenital lipodystrophy.

7. The method of claim 6, wherein the congenital lipodystrophy is congenital generalized lipodystrophy (Beradinelli-Seip syndrome) or familial partial lipodystrophy.

8. The method of claim 1, wherein the FGF-1 compound is administered intravenously.

9. The method of claim 1, wherein the FGF-1 compound is administered subcutaneously.

10. The method of claim 1, wherein the FGF-1 compound is administered in combination with at least one additional therapeutic compound.

11. The method of claim 1, wherein the FGF-1 compound comprises an FGF-1 protein comprising at least 80%, at least 85%, at least 90%, at least 90%, at least 98%, or at least 99% sequence identity to SEQ ID NO: 2 or 3.

12. The method of claim 1, wherein the FGF-1 compound comprises a functional fragment of an FGF-1 protein.

13. The method of claim 12, wherein the functional fragment of the FGF-1 protein comprises a mature form of FGF-1.

14. The method of claim 12, wherein the functional fragment of the FGF-1 protein comprises a functional fragment of SEQ ID NO: 3.

15. The method of claim 1, wherein the therapeutically effective amount is between about 0.01 to about 50 mg per kg body weight.

16. The method of claim 1, wherein the therapeutically effective amount of the FGF-1 compound is administered daily, twice daily, every other day, bi-weekly, weekly, or monthly.

17. The method of claim 2, wherein the antiretroviral agent and the FGF-1 compound are administered simultaneously.

18. The method of claim 2, wherein the antiretroviral agent and the FGF-1 compound are administered separately.

19. The method of claim 2, wherein the antiretroviral agent and the FGF-1 compound are administered sequentially.

20. The method of claim 1, wherein the individual is a human.


SEQUENCE LISTING (3 sequences)

SEQ ID NO: 1 - DNA, 468 bp, Homo sapiens
  CDS (1)..(468)
atggctgaaggggaaatcaccaccttcacagccctgaccgagaagtttaatctgcctccagggaattacaagaagcccaaactcctctactgtagcaacgggggccacttcctgaggatccttccggatggcacagtggatgggacaagggacaggagcgaccagcacattcagctgcagctcagtgcggaaagcgtgggggaggtgtatataaagagtaccgagactggccagtacttggccatggacaccgacgggcttttatacggctcacagacaccaaatgaggaatgtttgttcctggaaaggctggaggagaaccattacaacacctatatatccaagaagcatgcagagaagaattggtttgttggcctcaagaagaatgggagctgcaaacgcggtcctcggactcactatggccagaaagcaatcttgtttctccccctgccagtctcttctgattaa

SEQ ID NO: 2 - protein, 155 aa, Homo sapiens
MAEGEITTFTALTEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 3 - protein, 140 aa, Homo sapiens
FNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD